(19) United States
(12) Patent Application Publication
(10) Pub. No.: US 2022/0272935 A1
(43) Pub. Date: Sep. 1, 2022

(54) Novel Xanthomonas Strains and Related Methods

(71) Applicants: Agriculture Victoria Services PTY LTD, Bundoora, Victoria (AU); Dairy Australia Limited, Southbank, Victoria (AU); Geoffrey Gardiner Dairy Foundation Limited, Melbourne, Victoria (AU)

(72) Inventors: Tongda Li, Southbank (AU); Ian Ross Tannenbaum, Bundoora (AU); Jatinder Kaur, Taylors Hill (AU); Christian Krill, Reservoir (AU); Timothy Ivor Sawbridge, Coburg (AU); Ross Mann, Coburg (AU); German Carlos Spangenberg, Bundoora (AU)

(73) Assignees: Agriculture Victoria Services PTY LTD, Bundoora, Victoria (AU); Dairy Australia Limited, Southbank, Victoria (AU); Geoffrey Gardiner Dairy Foundation Limited, Melbourne, Victoria (AU)

(21) Appl. No.: 17/627,796

(22) PCT Filed: Jul. 17, 2020
(86) PCT No.: PCT/AU2020/050738

§ 371 Date: Jan. 17, 2022

(30) Foreign Application Priority Data
Jul. 19, 2019 (AU) 2019902562

(51) Int. Cl. A01H 17/00; A01H 5/10; C12N 15/10; G16B 30/10

(52) U.S. Cl. CPC A01H 17/00; A01H 5/10; C12N 15/1058; C12N 15/1079; G16B 30/10

(57) ABSTRACT

The present invention relates to an endophyte strain isolated from a plant of the Poaceae family, wherein said endophyte is a strain of Xanthomonas sp. which provides bioprotection and/or biofertilizer phenotypes to plants into which it is inoculated. The present invention also discloses plants infected with the endophyte and related methods.

### FIELD OF THE INVENTION

The present invention relates to novel plant microbiome strains, plants infected with such strains and related methods.

### BACKGROUND OF THE INVENTION

Microbes represent an invaluable source of novel genes and compounds that have the potential to be utilised in a range of industrial sectors. Scientific literature gives numerous accounts of microbes being the primary source of antibiotics, immune-suppressants, anticancer agents and cholesterol-lowering drugs, in addition to their use in environmental decontamination and in the production of food and cosmetics.

A relatively unexplored group of microbes known as endophytes, which reside e.g. in the tissues of living plants, offer a particularly diverse source of novel compounds and genes that may provide important benefits to society, and in particular, agriculture.

Endophytes may be fungal or bacterial. Endophytes often form mutualistic relationships with their hosts, with the endophyte conferring increased fitness to the host, often through the production of defence compounds. At the same time, the host plant offers the benefits of a protected environment and nutriment to the endophyte.

Important forage grasses perennial ryegrass (Lolium perenne) are commonly found in association with fungal and bacterial endophytes. However, there remains a general lack of information and knowledge of the endophytes of these grasses as well as of methods for the identification and characterisation of novel endophytes and their deployment in plant improvement programs.

Knowledge of the endophytes of perennial ryegrass may allow certain beneficial traits to be exploited in enhanced pastures, or lead to other agricultural advances, e.g. to the benefit of sustainable agriculture and the environment.

There exists a need to overcome, or at least alleviate, one or more of the difficulties or deficiencies associated with the prior art.

### SUMMARY OF THE INVENTION

In one aspect, the present invention provides a substantially purified or isolated endophyte strain isolated from a plant of the Poaceae family, wherein said endophyte is a strain of Xanthomonas sp. which provides bioprotection and/or biofertilizer phenotypes to plants into which it is inoculated. In a preferred embodiment, the Xanthomonas sp. strain may be a strain selected from the group consisting of GW, SS and SI as described herein and as deposited with The National Measurement Institute of 1/153 Bertie Street, Port Melbourne, VIC 3207, Australia on 17 May 2019 with accession numbers V19/009902, V19/009905 and V19/009909, respectively.

As used herein the term “endophyte” is meant a bacterial or fungal strain that is closely associated with a plant. By “associated with” in this context is meant that the bacteria or fungus lives on, in or in close proximity to a plant. For example, it may be endophytic, for example living within the internal tissues of a plant, or epiphytic, for example growing externally on a plant.

As used herein the term “substantially purified” is meant that an endophyte is free of other organisms. The term includes, for example, an endophyte in axenic culture. Preferably, the endophyte is at least approximately 90% pure, more preferably at least approximately 95% pure, even more preferably at least approximately 98% pure, even more preferably at least approximately 99% pure.

As used herein the term ‘isolated’ means that an endophyte is removed from its original environment (e.g. the natural environment if it is naturally occurring). For example, a naturally occurring endophyte present in a living plant is not isolated, but the same endophyte separated from some or all of the coexisting materials in the natural system, is isolated.

As used herein the term “bioprotection and/or biofertilizer” means that the endophyte possesses genetic and/or metabolic characteristics that result in a beneficial phenotype in a plant harbouring, or otherwise associated with, the endophyte. Such beneficial properties include improved resistance to pests and/or diseases, improved tolerance to water and/or nutrient stress, enhanced biotic stress tolerance, enhanced drought tolerance, enhanced water use efficiency, reduced toxicity and enhanced vigour in the plant with which the endophyte is associated, relative to an organism not harboring the endophyte or harboring a control endophyte such as standard toxic (ST) endophyte.

The pests and/or diseases may include, but are not limited to, fungal and bacterial pathogens. In a particularly preferred embodiment, the endophyte may result in the production of the bioprotectant compound in the organism with which it is associated.

As used herein, a bioprotectant compound is meant as a compound that provides bioprotection to the plant or aids the defense of the plant with which it is associated against pests and/or diseases, such as fungal and/or bacterial pathogens. A bioprotectant compound may also be known as a ‘biocidal compound’.

In a particularly preferred embodiment, the endophyte produces a bioprotectant compound and provides bioprotection to the organism against fungal and/or bacterial pathogens. The terms bioprotectant, bioprotective and bioprotection (or any other variations) may be used interchangeably herein.

Thus, in a preferred embodiment, the present invention provides a method of providing bioprotection to a plant against bacterial and/or fungal pathogens, said method including infecting the plant with an endophyte as hereinbefore described and cultivating the plant.

In a particularly preferred embodiment the bioprotectant compound is selected from the group consisting of siderophore xanthoferrin, and/or xanthomonadin, or a derivative, isomer and/or salt thereof.

The endophyte may be suitable as a biofertilizer to improve the availability of nutrients to the plant with which the endophyte is associated, including but not limited to improved tolerance to nutrient stress.

Thus, in a preferred embodiment, the present invention provides a method of providing biofertilizer to a plant, said method including infecting the plant with an endophyte as hereinbefore described and cultivating the plant.

The nutrient stress may be lack of or low amounts of a nutrient such as phosphate and/or nitrogen. The endophyte may be capable of growing in conditions such as low nitrogen and/or low phosphate and enable these nutrients to be available to the plant with which the endophyte is associated.

The endophyte may result in the production of organic acids and/or the solubilisation of phosphate in the organism with which it is associated and/or provide a source of phosphate to the plant.

Alternatively, or in addition, the endophyte may be capable of nitrogen fixation. Thus, if an endophyte is capable of nitrogen fixation, the plant with which the endophyte is associated may be capable of growing in low nitrogen conditions and/or the endophyte may provide a source of nitrogen to the plant.

In a particularly preferred embodiment, the endophyte provides the ability of the organism to grow in low nitrogen.

As used herein the term “plant of the Poaceae family” is a grass species, particularly a pasture grass such as ryegrass (Lolium) or fescue (Festuca), more particularly perennial ryegrass (Lolium perenne L.) or tall fescue (Festuca arundinaceum, otherwise known as Lolium arundinaceum).

In another aspect, the present invention provides a plant or part thereof infected with an endophyte as hereinbefore described. In preferred embodiments, the plant or part thereof infected with the endophyte may produce a bioprotectant compound. Preferably, the bioprotectant compound is selected from siderophore xanthoferrin and xanthomonadin.

Also in preferred embodiments, the plant or part thereof includes an endophyte-free host plant or part thereof stably infected with said endophyte.

The plant inoculated with the endophyte may be a grass or non-grass plant suitable for agriculture, specifically a forage, turf, or bioenergy grass, or a grain crop or industrial crop.

Preferably, the plant is a grass species plant, specifically a forage, turf, bioenergy, grain crop or industrial crop grass.

The forage, turf or bioenergy grass may be those belonging to the Brachiaria-Urochloa species complex (panic grasses), including Brachiaria brizantha, Brachiaria decumbens, Brachiaria humidicola, Brachiaria stolonifera, Brachiaria ruziziensis, B. dictyoneura, Urochloa brizantha, Urochloa decumbens, Urochloa humidicola, Urochloa mosambicensis as well as interspecific and intraspecific hybrids of Brachiaria-Urochloa species complex such as interspecific hybrids between Brachiaria ruziziensis x Brachiaria brizantha, Brachiaria ruziziensis x Brachiaria decumbens, [Brachiaria ruziziensis x Brachiaria decumbens ]x Brachiaria brizantha, [Brachiaria ruziziensis x Brachiaria brizantha]x Brachiaria decumbens.

The forage, turf or bioenergy grass may also be those belonging to the genera Lolium and Festuca, including L. perenne (perennial ryegrass) and L. arundinaceum (tall fescue) and L. multiflorum (Italian ryegrass).

The grain crop or industrial crop may be a non-grass species, for example, any of soybeans, cotton and grain legumes, such as lentils, field peas, fava beans, lupins and chickpeas, as well as oilseed crops, such as canola.

Thus, the grain crop or industrial crop species may selected from the group consisting of wheat, barley, oats, chickpeas, triticale, fava beans, lupins, field peas, canola, cereal rye, vetch, lentils, millet/panicum, safflower, linseed, sorghum, sunflower, maize, canola, mungbeans, soybeans, and cotton.

The grain crop or industrial crop grass may be those belonging to the genus Triticum, including T. aestivum (wheat), those belonging to the genus Hordeum, including H. vulgare (barley), those belonging to the genus Avena, including A. sativa (oats), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), and those belonging to the genera Miscanthus, Paspalum, Pennisetum, Poa, Eragrostis and Agrostis.

A plant or part thereof may be infected by a method selected from the group consisting of inoculation, breeding, crossing, hybridisation, transduction, transfection, transformation and/or gene targeting and combinations thereof.

Without wishing to be bound by theory, it is believed that the endophyte of the present invention may be transferred through seed from one plant generation to the next. The endophyte may then spread or locate to other tissues as the plant grows, i.e. to roots. Alternatively, or in addition, the endophyte may be recruited to the plant root, e.g. from soil, and spread or locate to other tissues.

Thus, in a further aspect, the present invention provides a plant, plant seed or other plant part derived from a plant or part thereof as hereinbefore described. In preferred embodiments, the plant, plant seed or other plant part may produce a bioprotectant compound. Preferably, the bioprotectant compound is selected from siderophore xanthoferrin and xanthomonadin or derivative, isomer and/or salt thereof.

In another aspect, the present invention provides the use of an endophyte as hereinbefore described to produce a plant or part thereof stably infected with said endophyte. The present invention also provides the use of an endophyte as hereinbefore described to produce a plant or part thereof as hereinbefore described.

In another aspect, the present invention provides a bioprotectant compound produced by an endophyte as hereinbefore described, or a derivative, isomer and/or a salt thereof. Preferably, the bioprotectant compound is selected from siderophore xanthoferrin and xanthomonadin.

The bioprotectant compound may be produced by the endophyte when associated with a plant, e.g. a plant of the Poaceae family as described above.

Thus, in another aspect, the present invention provides a method for producing a bioprotectant compound, said method including infecting a plant with an endophyte as hereinbefore described and cultivating the plant under conditions suitable to produce the bioprotectant compound. The endophyte-infected plant or part thereof may be cultivated by known techniques. The person skilled in the art may readily determine appropriate conditions depending on the plant or part thereof to be cultivated. Preferably, the bioprotectant compound is selected from siderophore xanthoferrin and xanthomonadin or derivative, isomer and/or salt thereof.

The bioprotectant compound may also be produced by the endophyte when it is not associated with a plant. Thus, in yet another aspect, the present invention provides a method for producing a bioprotectant compound, said method including culturing an endophyte as hereinbefore described, under conditions suitable to produce the bioprotectant compound. Preferably, the bioprotectant compound is selected from siderophore xanthoferrin and xanthomonadin or derivative, isomer and/or salt thereof.

The conditions suitable to produce the bioprotectant compound may include a culture medium including a source of carbohydrates. The source of carbohydrates may be a starch/sugar-based agar or broth such as potato dextrose agar, potato dextrose broth or half potato dextrose agar or a cereal-based agar or broth such as oatmeal agar or oatmeal broth. Other sources of carbohydrates may include endophyte agar, Murashige and Skoog with 20% sucrose, half V8 juice/half PDA, water agar and yeast malt extract agar. The endophyte may be cultured under aerobic or anaerobic conditions and may be cultured in a bioreactor. Preferably, the bioprotectant compound is selected from siderophore xanthoferrin and xanthomonadin.

In a preferred embodiment of this aspect of the invention, the method may include the further step of isolating the bioprotectant compound from the plant or culture medium. Preferably, the bioprotectant compound is selected from siderophore xanthoferrin and xanthomonadin, or derivative, isomer and/or salt thereof.

The endophyte-infected plant or part thereof may be cultivated by known techniques. The person skilled in the art may readily determine appropriate conditions depending on the plant or part thereof to be cultivated.

The endophyte of the present invention may display the ability to solubilise phosphate.

In another aspect, the present invention provides a method of increasing phosphate use efficiency and/or increasing phosphate solubilisation by a plant, said method including infecting a plant with an endophyte as hereinbefore described, and cultivating the plant.

In yet another aspect, the present invention provides a method of reducing phosphate levels in soil, said method including infecting a plant with an endophyte as hereinbefore described, and cultivating the plant in the soil.

The endophyte of the present invention may be capable of nitrogen fixation. Thus, in yet another aspect, the present invention provides a method of growing the plant in low nitrogen containing medium, said method including infecting a plant with an endophyte as hereinbefore described, and cultivating the plant. Preferably, the low nitrogen medium is low nitrogen containing soil.

In yet a further aspect, the present invention provides a method of increasing nitrogen use efficiency or increasing nitrogen availability to a plant, said method including infecting a plant with an endophyte as hereinbefore described, and cultivating the plant.

In yet another aspect, the present invention provides a method of reducing nitrogen levels in soil, said method including infecting a plant with an endophyte as hereinbefore described, and cultivating the plant in the soil.

In a further aspect, the present invention provides a method of providing bioprotection to a plant against bacterial and/or fungal pathogens and/or providing biofertilizer to a plant, said method including infecting the plant with and endophyte as hereinbefore described. Preferably, the method includes providing bioprotection to the plant and includes production of a bioprotectant compound in the plant into which the endophyte is inoculated.

The endophyte-infected plant or part thereof may be cultivated by known techniques. The person skilled in the art may readily determine appropriate conditions depending on the plant or part thereof to be cultivated.

The production of a bioprotectant compound has particular utility in agricultural plant species, in particular, forage, turf, or bioenergy grass species, or grain crop species or industrial crop species. These plants may be cultivated across large areas of e.g. soil where the properties and biological processes of the endophyte as hereinbefore described and/or bioprotectant compound produced by the endophyte may be exploited at scale. Preferably, the bioprotectant compound is selected from siderophore xanthoferrin and xanthomonadin or derivative, isomer and/or salt thereof.

The part thereof of the plant may be, for example, a seed.

In preferred embodiments, the plant is cultivated in the presence of soil phosphate and/or nitrogen, alternatively or in addition to applied phosphate and/or nitrogen. The applied phosphate and/or applied nitrogen may be by way of, for example, fertiliser. Thus, preferably, the plant is cultivated in soil.

In preferred embodiments, the endophyte may be a Xanthomonas sp. strain selected from the group consisting of GW, SS and SI as described herein and as deposited with The National Measurement Institute of 1/153 Bertie Street, Port Melbourne, VIC 3207, Australia on 17 May 2019 with accession numbers V19/009902, V19/009905 and V19/009909, respectively.

Preferably, the plant is a forage, turf, bioenergy grass species or, grain crop or industrial crop species, as hereinbefore described.

The part thereof of the plant may be, for example, a seed.

In preferred embodiments, the plant is cultivated in the presence of soil phosphate and/or applied phosphate. The applied phosphate may be by way of, for example, fertiliser. Thus, preferably, the plant is cultivated in soil.

Alternatively, or in addition, the plant is cultivated in the presence of soil nitrogen and/or applied nitrogen. The applied nitrogen may be by way of, for example, fertiliser. Thus, preferably, the plant is cultivated in soil.

The present invention will now be more fully described with reference to the accompanying Examples and drawings. It should be understood, however, that the description following is illustrative only and should not be taken in any way as a restriction on the generality of the invention described above.

### BRIEF DESCRIPTION OF THE DRAWINGS/FIGURES

FIG. 1—16S Amplicon sequence of novel bacterial strain GW (SEQ ID NO.1).

FIG. 2—Phylogenetic analysis of the gyrase B gene from the Xanthomonas sp. novel bacterial strains GW, SS and SI, in comparison to 11 related Xanthomonas species and the outgroups Lysobacter enzymogenes and Pseudoxanthomonas suwonensis.

FIG. 3—Phylogeny of X. translucens pathovars and Xanthomonas sp. novel bacterial strains GW, SS and SI. This maximum-likelihood tree was inferred based on 97 genes conserved among 19 genomes. Values shown next to branches were the local support values calculated using 1000 resamples with the Shimodaira-Hasegawa test.

FIG. 4—Secondary metabolite biosynthesis gene clusters in the Xanthomonas sp. novel bacterial strains GW, SS and SI identified using antiSMASH (Weber et al. 2015). The gene clusters have sequence homology and structure to (A) the xanthoferrin gene cluster, (B) the xanthomonadin gene cluster and (C) an unknown gene cluster.

FIG. 5—Whole genome sequence comparison of the Xanthomonas sp. novel bacterial strains GW (middle), SI (top) and SS (bottom). The links between genome sequences indicated percentage similarity (from 70% to 100%). Genetic variations, including non-identical regions and insertions/deletions/inversions, suggested that the novel bacterial strains GW, SI and SS are genetically different. The star indicates the site of the genome of novel bacterial strain GW where the unique secondary metabolite biosynthesis gene cluster is located.

FIG. 6—Type I and Type III secretion systems of bacteria.

FIG. 7—Gene clusters of bacterial secretion systems in: A. the endophyte Xanthomonas sp. novel bacterial strain GW from perennial ryegrass, B. the pathogen Xanthomonas translucens pv. translucens from barley, and C. the pathogen Xanthomonas translucens pv. undulosa from wheat. Grey shading indicates presence of gene in gene cluster.

FIG. 8—Genome alignment of Xanthomonas sp. novel bacterial strain GW (the outer circle, greys) and X. t. pv. undulosa Xtu4699 (the inner circle, black). The colour of the outer circle represented the sequence identity (dark grey: 90% -100%; white: <90%). The locations of T3SS, T3Es and TALEs genes that were detected on the genome of X. t. pv. undulosa Xtu4699 are designated.

FIG. 9—Image of 5 day old seedlings inoculated with the Xanthomonas sp. novel bacterial strain GW and an untreated control.

FIG. 10—Average shoot length of barley seedlings inoculated with bacterial strains of Xanthomonas sp. (strain GVV) and non-Xanthomonads (Strain 1, 2, 3), and grown for 5 days. The * indicates significant difference in the mean at p 0.05 between the control and the bacterial strains.

FIG. 11—Average root length of barley seedlings inoculated with bacterial strains of Xanthomonas sp. (strain GW) and non-Xanthomonads (Strain 1, 2, 3), and grown for 5 days. The * indicates significant difference in the mean at p 0.05 between the control and the bacterial strains.

FIG. 12—Agarose gel electrophoresis (2% [w/v]) of PCR amplicons generated using the GW strain-specific primers on Xanthomonas sp. strains GW, SS, SI, a negative control (NC) and a 2 kb DNA molecular ladder (M)

FIG. 13—Average root length of barley seedlings inoculated with bacterial strains of Xanthomonas sp. (strain GVV) and non-Xanthomonads (Strain 1, 2, 3, 4), and grown for 4 days on nitrogen free media. The star indicates significant difference in the mean at p 0.05 between the control and the bacterial strains.

FIG. 14—Average shoot length of barley seedlings inoculated with bacterial strains of Xanthomonas sp. (strain GVV) and non-Xanthomonads (Strain 1, 2, 3, 4), and grown for 4 days on nitrogen free media. The star indicates significant difference in the mean at p 0.05 between the control and the bacterial strains.

FIG. 15—Average root length of barley seedlings inoculated with bacterial strains of Xanthomonas sp. (strain GVV) and non-Xanthomonads (Strain 1, 2, 3, 4), and grown for 4 days on media containing insoluble phosphate. The star indicates significant difference in the mean at p 0.05 between the control and the bacterial strains.

FIG. 16—Average shoot length of barley seedlings inoculated with bacterial strains of Xanthomonas sp. (strain GVV) and non-Xanthomonads (Strain 1, 2, 3, 4), and grown for 4 days on media containing insoluble phosphate. The star indicates significant difference in the mean at p 0.05 between the control and the bacterial strains.

FIG. 17—Average root and shoot length of barley seedlings inoculated with novel Xanthomonas sp. bacterial strain GW at different concentrations (10⁰, 10⁻¹, 10⁻²), and grown for 7 days.

### DETAILED DESCRIPTION OF THE EMBODIMENTS

Isolation and characterisation of plant associated Xanthomonas sp. novel bacterial strains providing bioprotection phenotypes to plants.

Three novel plant associated Xanthomonas sp. bacterial strains GW, SS and SI were isolated from perennial ryegrass (Lolium perenne) plants. They display the ability to inhibit the growth of plant fungal pathogens in plate assays. The genomes of the three novel Xanthomonas sp. bacterial strains have been sequenced and are shown to be novel species, related to other Xanthomonad bacteria including Xanthamonas translucens. Analysis of the genome sequence has shown that all three Xanthomonas bacterial strains do not contain the type III secretion system shown to be essential for pathogenesis in pathogenic strains but do contain a type IV secretion system that has been implicated in an endophytic life cycle. Although the bacterial strains are closely related they have differing biocidal activities, with one strain antagonistic to more fungi than the other strains. These bacterial strains have been used to inoculate barley (Hordeum vulgare) seeds under glasshouse conditions and have been demonstrated not to cause disease in these barley plants. These barley plants are also able to produce seed. Novel bacterial strain GW also enhances root growth in nitrogen limiting conditions and in insoluble phosphate. The optimal concentration of inoculum for novel bacterial strain GW is a dilution of an overnight culture (10⁻¹, 10⁻²). Overall, novel plant associated Xanthomonas sp. bacterial strains GW, SS and SI offer both bioprotectant and biofertilizer activity (GW only).

### Example 1

### Isolation of Bacterial Strains

### Seed Associated Bacterial Strains

Seeds from perennial ryegrass (Lolium perenne) were surface-sterilised by soaking in 80% ethanol for 3 mins, then washing 5 times in sterile distilled water. The seeds were then plated onto sterile filter paper soaked in sterile water in sterile petri dishes. These plates were stored at room temperature in the dark to allow seedlings to germinate for 1-2 weeks. Once the seedlings were of sufficient size, the plants were harvested. In harvesting, the remaining seed coat was discarded, and the aerial tissue and root tissue were harvested. The plant tissues were submerged in sufficient Phosphate Buffered Saline (PBS) to completely cover the plant tissue, and ground using a Qiagen TissueLyser II, for 1 minute at 30 Hertz. A 10 μl aliquot of the macerate was added to 90 μl of PBS. Subsequent 1 in 10 dilutions of the 10⁻¹ suspension were used to create additional 10⁻² to 10⁻⁴ suspensions.

Once the suspensions were well mixed 50 μl aliquots of each suspension were plated onto Reasoners 2 Agar (R2A) for growth of bacteria. Dilutions that provided a good separation of bacterial colonies were subsequently used for isolation of individual bacterial colonies through re-streaking of single bacterial colonies from the dilution plates onto single R2A plates to establish a pure bacterial colony.

### Mature Plant Associated Bacterial Strains

Leaf and root tissue were harvested from mature plants grown in the field or grown in pots in a greenhouse. Root tissue was washed in PBS buffer to remove soil particles and sonicated (10 mins) to remove the rhizosphere. The harvested tissues were placed into sufficient PBS to completely cover the tissue and processed as per the previous section to isolate pure bacterial cultures.

Around 300 bacterial strains were obtained from sterile seedlings, and 300 strains from mature plants. The novel bacterial strain GW was collected from seed of perennial ryegrass, while SS and SI were collected from mature plants.

### Example 2

Identification of Xanthomonas sp. Novel Bacterial Strain
Amplicon (16S rRNA Gene) Sequencing

A phylogenetic analysis of the novel bacterial strain GW was undertaken by sequence homology comparison of the 16S rRNA gene. The novel bacterial strain GW was grown overnight in Reasoners 2 Broth (R2B) media. DNA was extracted from pellets derived from the overnight culture using a DNeasy Blood and Tissue kit (Qiagen) according to manufacturer's instructions. The 16S rRNA gene amplification used the following PCR reagents: 14.8 μL H₂O, 2.5 μL 10× reaction buffer, 0.5 μL 10 mM dNTPs, 2.5 μL each of the 5 μM 27F primer (5′- AGAGTTTGATCMTGGCTCAG -3′) (SEQ ID NO. 2) and 5 μM reverse primers 1492R (5′- GGTTACCTTGTTACGACTT -3′) (SEQ ID NO. 3), 0.2 μL of Immolase enzyme, and template to a final volume of 25 μL. The PCR reaction was then run in an Agilent Surecycler 8800 (Applied Biosystems) with the following program; a denaturation step at 94° C. for 15 min; 35 cycles of 94° C. for 30 sec, 55° C. for 10 sec, 72° C. 1 min; and a final extension step at 72° C. for 10 min.

Shrimp alkaline phosphatase (SAP) exonuclease was used to purify the 16S rRNA gene PCR amplicon. The SAP amplicon purification used the following reagents: 7.375 μL H₂O, 2.5 μL 10× SAP, and 0.125 μL Exonuclease I. The purification reaction was incubated at 37° C. for 1 hr, followed by 15 min at 80° C. to deactivate the exonuclease.

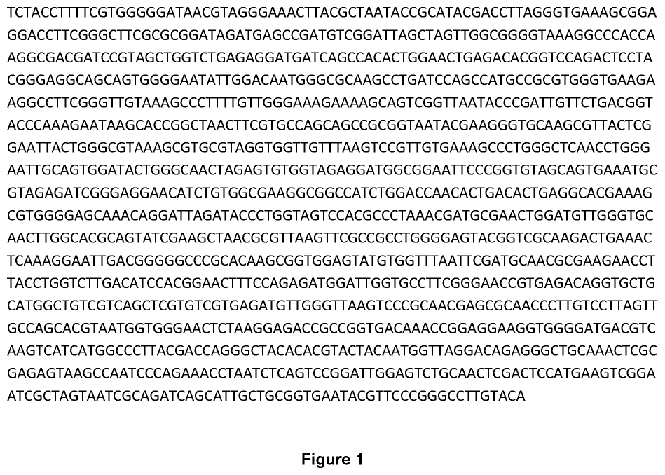

The purified 16S rRNA gene amplicon was sequenced using the BigDye® Terminator v3.1 Cycle Sequencing Kit (Thermofisher) with the following reagents; 10.5 μL H₂O, 3.5 μL 5× Seq buffer, 0.5 μL BigDye®, 2.5 μL of either the 3.2 μM Forward (27F) and 3.2 μM Reverse primers (1492R), and 4.5 μL of PCR amplicon as template, to a final reaction volume of 20 μL. The sequencing PCR reaction was then run in an Agilent Surecycler 8800 (Applied Biosystems) with the following program; denaturation step at 94° C. for 15 min; followed by 35 cycles of 94° C. for 30 sec, 55° C. for 10 sec, 72° C. 1 min; and one final extension step at 72° C. for 10 min. The 16S rRNA gene amplicon from novel bacterial strain GW was sequenced on an AB13730XL (Applied Biosystems). A 1269 bp 16S rRNA gene sequence was generated (FIG. 1). The sequence was aligned by BLASTn on NCBI against the non-redundant nucleotide database and the 16S ribosomal RNA database.

BLASTn Hit Against Database nr; Xanthomonas sp. Strain PRd6 16S Ribosomal RNA Gene, Partial Sequence

| | Total | Query | | | |
|---|---|---|---|---|---|
| Max Score | Score | Coverage | E-Value | % Identity | Accession |
| 2289 | 2289 | 100% | 0 | 100.00% | KY203971.1 |

BLASTn Hit Against Database 16S Ribosomal RNA; Xanthomonas translucens Strain

XT 2 16S Ribosomal RNA Gene, Partial Sequence

| | Total | Query | | | |
|---|---|---|---|---|---|
| Max Score | Score | Coverage | E-Value | % Identity | Accession |
| 2271 | 2271 | 100% | 0 | 99.68% | NR_036968.1 |

The preliminary taxonomic identification of the novel bacterial strain GW was a novel Xanthomonas sp., closely related to Xanthomonas transluscens.

### Genomics

The genome of the novel bacterial strain GW was sequenced, along with two additional Xanthomonas strains SS and SI. These novel bacterial strains were retrieved from the glycerol collection stored at −80° C. by streaking on R2A plates. Single colonies from these plates were grown overnight in Nutrient Broth and pelleted. These pellets were used for genomic DNA extraction using the bacteria protocol of Wizard® Genomic DNA Purification Kit (A1120, Promega). DNA sequencing libraries were generated for Illumina sequencing using the Illumina Nextera XT DNA library prep protocol. All libraries were sequenced using an Illumina MiSeq platform or HiSeq platform. Raw reads from the sequencer were filtered to remove any adapter and index sequences as well as low quality bases using Trimmomatic (Bolger, Lohse & Usadel 2014) with the following options: ILLUMINACLIP: NexteraPE-PE.fa:2:30:10 LEADING:3 TRAILING:3 SLIDINGWINDOW:4:15 MINLEN:36. To enable full genome assembly, long reads were generated for the three Xanthomonas sp. novel bacterial strain by sequencing DNA using Oxford Nanopore Technologies (ONT) MinION platform. The DNA from the Wizard® Genomic DNA Purification Kit was first assessed with the genomic assay on Agilent 2200 TapeStation system (Agilent Technologies, Santa Clara, Calif., USA) for integrity (average molecular weight ≥30 Kb). The sequencing library was prepared using an in-house protocol modified from the official protocols for transposases-based library preparation kits (SQK-RAD004/SQK-RBK004, ONT, Oxford, UK). All libraries were sequenced on a MinION Mk1B platform (MIN-101B) with R9.4 flow cells (FLO-MIN106) and under the control of MinKNOW software. After the sequencing run finished, the fast5 files that contain raw read signals were transferred to a separate, high performance computing Linux server for local basecalling using ONT's Albacore software (Version 2.3.1) with default parameters. For libraries prepared with the barcoding kit (SQK-RBK004), barcode demultiplexing was achieved during basecalling. The sequencing summary file produced by Albacore was processed by the R script minion qc (https://github.com/roblanf/minion_qc) and NanoPlot (De Coster et al. 2018) to assess the quality of each sequencing run, while Porechop (Version 0.2.3, https://github.com/rrwick/Porechop) was used to remove adapter sequences from the reads. Reads which were shorter than 300 bp were removed and the worst 5% of reads (based on quality) were discarded by using Filtlong (Version 0.2.0, https://gitthub.com/rrwick/Filtlong).

The whole genome sequence of the three Xanthomonas sp. novel bacterial strains were assembled using Unicycler (Wick et al. 2017). Unicycler performed hybrid assembly when both Illumina reads and MinION reads were available. MinION reads were mainly used to resolve repeat regions in the genome, whereas Illumina reads were used by Pilon (Walker et al. 2014) to correct small base-level errors. Multiple rounds of Racon (Vaser et al. 2017) polishing were then carried out to generate consensus sequences. Assembly graphs were visualised by using Bandage (Wick et al. 2015).

A complete circular chromosome sequence was produced for the three Xanthomonas sp. novel bacterial strains. The genome size for the novel bacterial strains GW, SS and SI were 5,233,349 bp, 5,185,085 bp and 5,246,417 bp respectively (Table 1). The percent GC content ranged from 68.37% -68.55%. These novel bacterial strains were annotated by Prokka (Seemann 2014) with a custom, genus-specific protein database to predict genes and corresponding functions, which were then screened manually to identify specific traits.

The number of genes for the novel bacterial strains GW, SS and SI were 4,425, 4,291 and 4,290 genes respectively (Table 2).

TABLE 1
Summary of properties of the final genome sequence assembly

| | Genome size | GC content | Coverage | Coverage |
|---|---|---|---|---|
| Strain ID | (bp) | (%) | Illumina reads | ONT MinION |
| GW | 5,233,349 | 68.37 | 105.6x | 97x |
| SS | 5,185,085 | 68.55 | 1167.9x | 23.7x |
| SI | 5,246,417 | 68.44 | 584.5x | 24.9x |

TABLE 2
Summary of genome coding regions

| | Genome size | No. of | No. of | No. of | No. of | No. of |
|---|---|---|---|---|---|---|
| Strain ID | (bp) | tRNA | tmRNA | rRNA | CDS | gene |
| GW | 5,233,349 | 60 | 1 | 6 | 4358 | 4425 |
| SS | 5,185,085 | 57 | 1 | 6 | 4227 | 4291 |
| SI | 5,246,417 | 63 | 1 | 6 | 4290 | 4360 |

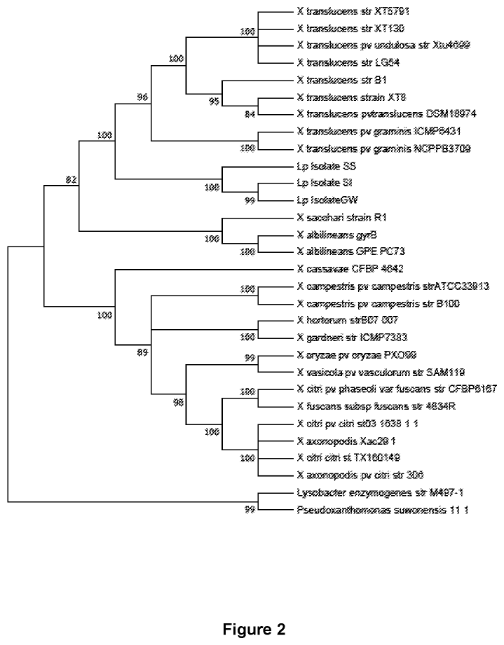

The gyrase B gene was extracted from the genome sequences of the Xanthomonas sp. novel bacterial strains GW, SS and SI, and a multiple sequence alignment was performed with 20 gyrase B genes from X. translucens (9 strains), X. sacchari (1), X. albilineans (2), X. cassavae (1), X. campestris (2), X. hortorum (1), X. gardeneri, X. oryzae, X. vasicola (1), X. citri (2), X. axonopodis (2) and the outgroups Lysobacter enzymogenes and Pseudoxanthomonas suwonensis. A neighbour joining tree was generated from this alignment with 100 bootstraps performed (FIG. 2). The strains GW, SS and SI formed a distinct clade from X. translucens and X. sacchari and X. albilineans strains, which supports that these three strains are from a novel Xanthomonas species.

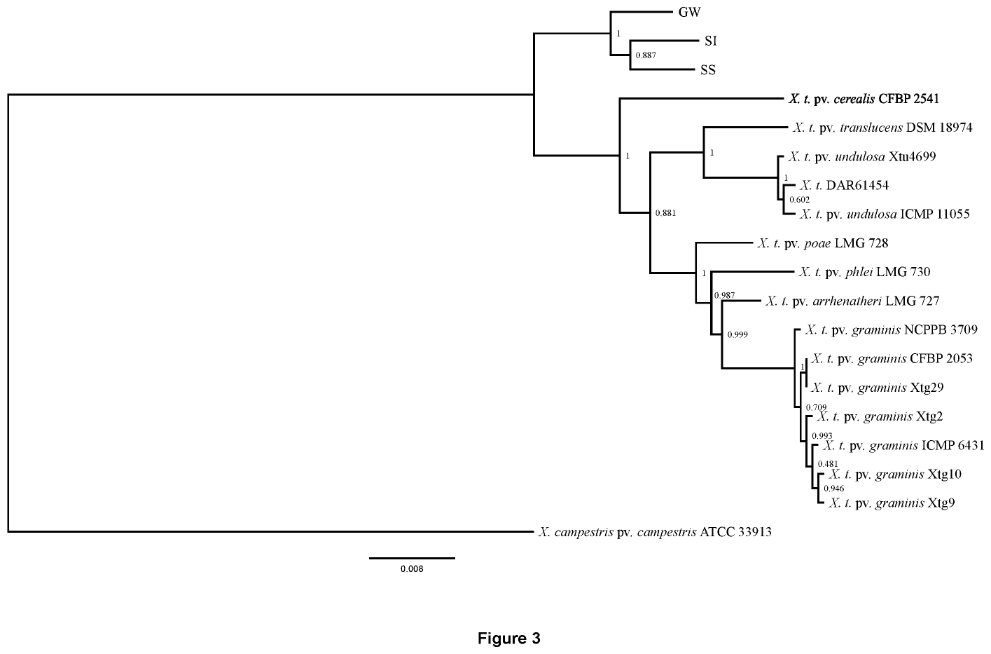

Fifteen X. translucens genome sequences and one X. campestris genome sequence that are publicly available on NCBI were acquired and used for pan-genome/comparative genome sequence analysis alongside Xanthomonas sp. novel bacterial strains GW, SS and SI. A total of 97 genes that are shared by all 19 strains were identified by running Roary (Page et al. 2015). PRANK (Löytynoja 2014) was then used to perform a codon aware alignment. A maximum-likelihood phylogenetic tree (FIG. 3) was inferred using FastTree (Price, Dehal & Arkin 2010) with Jukes-Cantor Joins distances and Generalized Time-Reversible and CAT approximation model. Local support values for branches were calculated using 1000 resamples with the Shimodaira-Hasegawa test. The novel bacterial strains GW, SS and SI clustered tightly together, suggesting a close phylogenetic relationship between these bacterial strains. Moreover, this cluster was separated from other X. translucens pathovars. with strong local support value (100%). This separation supports that these three bacterial strains are from a novel Xanthomonas species, but closely related to X. translucens pathovars.

### Example 3

Bioprotection Activity (In Vitro) of Xanthomonas sp. Strains

In vitro bioassays were established to test the bioactivity of the Xanthomonas sp. novel bacterial strains GW, SS and SI against five plant pathogenic fungi (Table 3). An unrelated bacterial strain (Strain X) was used as a negative control. The fungal pathogens were all isolated from monocot species, and were obtained from the National Collection of Fungi (Herbarium VPRI) and the AVR collection. Each bacterial strain was cultured in Nutrient Broth (BD Biosciences) overnight at 28° C. in a shaking incubator (200 rpm). Each bacterial strain was drop-inoculated (20 μL) onto four equidistant points on a Nutrient Agar (BD Biosciences) plate, which was then incubated overnight at 28° C. A 6 mm×6 mm agar plug of actively growing mycelia from the pathogen was placed at the centre of the plate. The bioassay was incubated for at least 5 days at 28° C. in the dark, and then the diameter of the fungal colony on the plate was recorded. For each treatment three plates were prepared as biological triplicates. OriginPro 2018 (Version b9.5.1.195) was used to carry out One-way ANOVA and Tukey Test to detect the presence of any significant difference (p≤0.05) between treatments.

TABLE 3
Pathogens used in the bioprotection bioassay.

| VPRI | | Host | | |
|---|---|---|---|---|
| Accession | | Taxonomic | | Collection |
| No. | Taxonomic Details | Details | State | Date |
| 12962 | Drechslera brizae (Y. Nisik.) | Briza maxima L. | Vic. | 24 Oct. 1985 |
| | Subram. & B. L. Jain | | | |
| 32148 | Sclerotium rolfsii Sacc. | Poa annua L. | Vic. | 1 Jan. 2005 |
| 42586a | Fusarium verticillioides | Zea mays L. | Vic. | 27 Feb. 2015 |
| | (Sacc.) Nirenberg | | | |
| 42563 | Bipolaris gossypina | Brachiaria | Qld | |
| N/A | Microdochium nivale | Lolium perenne L. | Vic | |

The Xanthomonas sp. novel bacterial strain GW inhibited the growth of all five pathogens, indicating it had broad spectrum biocidal activity, unlike novel bacterial strains SS and SI that only inhibited the growth of three and four pathogens respectively (Table 4, grey shading). Novel bacterial strain GW significantly inhibited the growth of Sclerotium rolfsii (74.80%) in comparison to Strain X, Microdochium nivale (67.87%) compared to bacterial strains SS, SI and X, and Bipolaris gossypina (54.67%), compared to bacterial strains SS, SI.

### Example 4

Genome Sequence Features Supporting the Bioprotection Niche of the Xanthomonas sp. Novel Bacterial Strains

### Secondary Metabolite Biosynthesis Gene Clusters

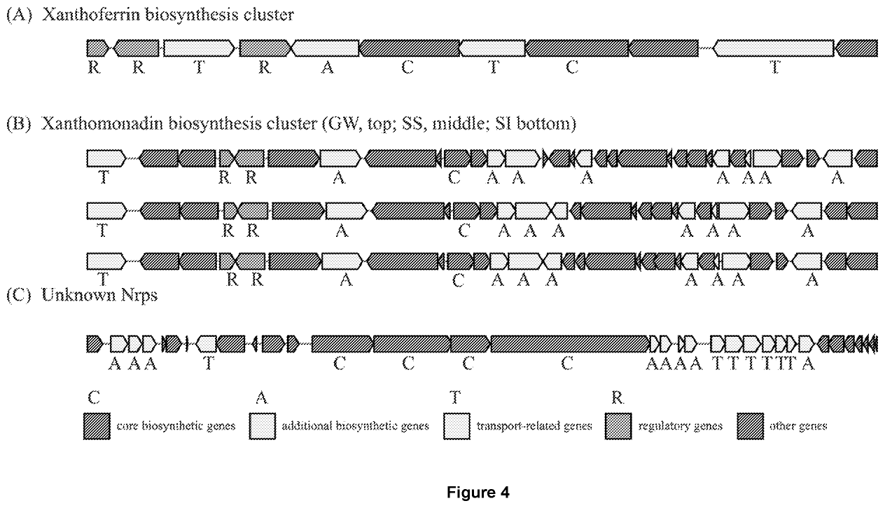

The genome sequences of the three Xanthomonas sp. novel bacterial strains GW, SS and SI were assessed for the presence of features associated with bioprotection. The annotated genome sequences were analysed by antiSMASH (Weber et al. 2015) to identify secondary metabolite biosynthesis gene clusters that are commonly associated with the production of biocidal compounds that aid in their defense. Annotated genome sequences were passed through antiSMASH with the following options: -clusterblast -asf -knownclusterblast -subclusterblast -smcogs -full-hmmer. A total of three secondary metabolite gene clusters were identified in the genome sequences of the three Xanthomonas sp. novel bacterial strains (FIG. 4). A biosynthetic gene cluster was identified in all three novel bacterial strains that had sequence homology and structure to the xanthoferrin gene cluster that produces the bioprotectant siderophore xanthoferrin (FIG. 4A). This gene cluster had the non-ribosomal peptide synthases (NRPS) essential for the biosynthesis of the nonribosomal peptide xanthoferrin and was identical in structure across the strains. A biosynthetic gene cluster was also shared by all three novel bacterial strains that had sequence homology and structure to the xanthomonadin gene cluster that produces the bioprotectant pigment xanthomonadin (FIG. 4B). This gene cluster had the polyketide synthase (PKS) essential for the biosynthesis of the polyketide xanthomonadin, but the cluster had slight variations in structure across three novel bacterial strains. A gene cluster was identified that was unique to novel bacterial strain GW, and had a NRPS essential for biosynthesis a nonribosomal peptide, but showed no sequence homology to other gene clusters in the antiSMASH database (FIG. 4C). This gene cluster is of interest as it may be linked to the biosynthesis of a compound that explains the biocidal activity of novel bacterial strain GW.

### Genome Sequence Alignment

The genome sequences of the novel bacterial strains GW, SS and SI were aligned using

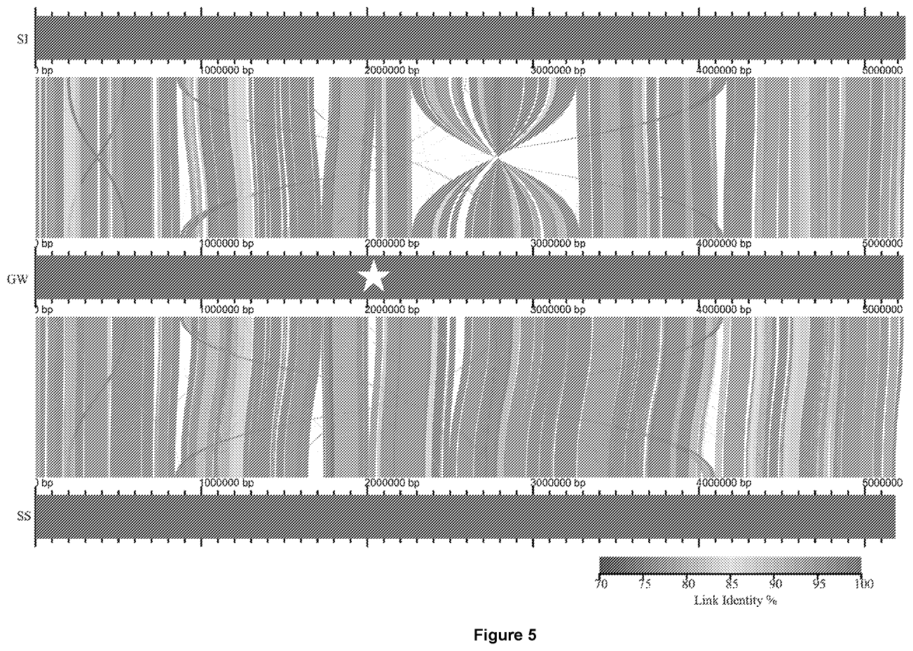

LASTZ (Version 1.04.00, http://www.bx.psu.edu/˜rsharris/lastz/) and visualised using AliTV (Ankenbrand et al. 2017) to validate the absence of the unique secondary metabolite gene cluster from novel bacterial strains SS and SI. A region of the genome of novel bacterial strain GW was identical between bases 1,997,794 and 2,067,075 that contained the unique secondary metabolite gene cluster, but was absent from novel bacterial strains SS and SI (FIG. 5, star).

### Example 5

Genome sequence Features Supporting the Endophytic Niche of the Xanthomonas sp. Novel Bacterial Strains

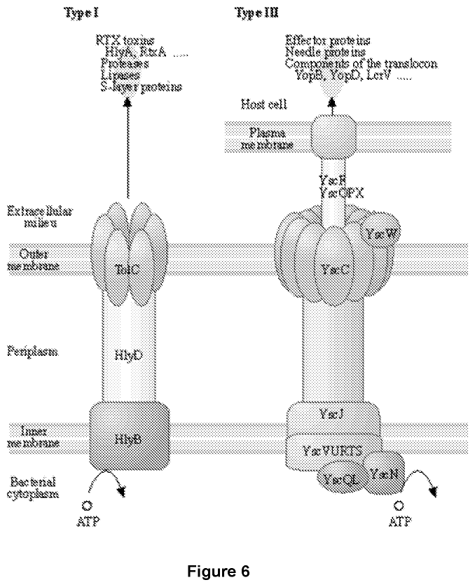

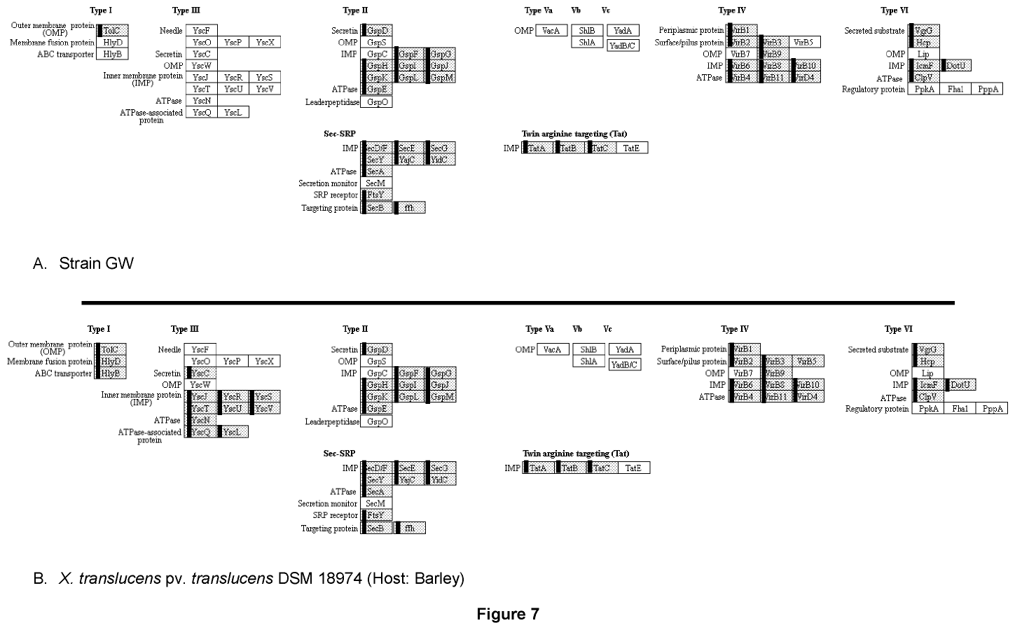

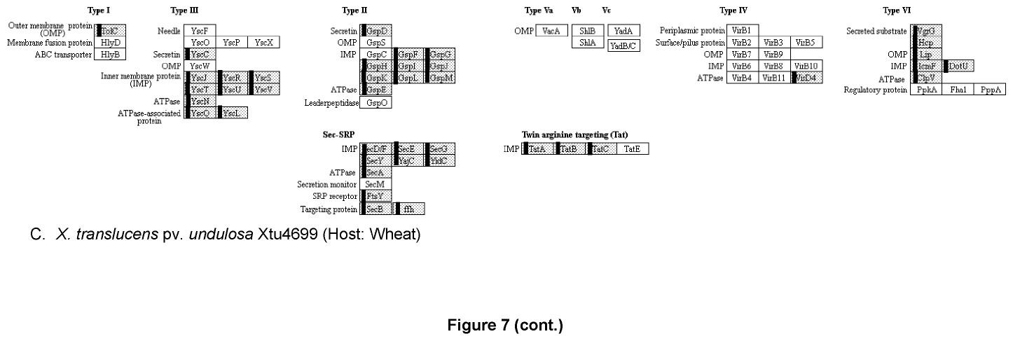

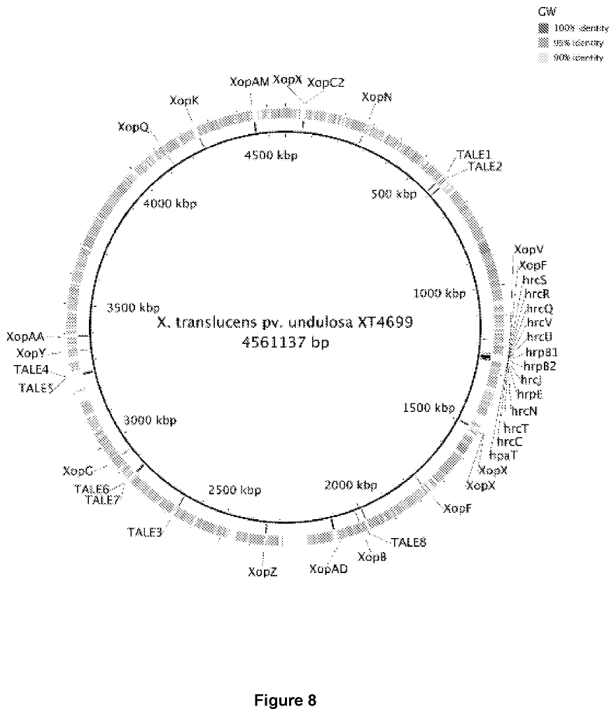

There have been nine virulence-related gene clusters identified in the X. translucens genome that are important for the pathogenicity of this species (Table 5). These include gene clusters that regulate biosynthesis of secretion systems (T1, T2, T3 and T6), pili (Type 4), flagella, xanthan and lipopolysaccharides (Table 5). The presence of these clusters in the genome of the Xanthomonas sp. novel bacterial strain GW was assessed through homology searches of gene sequences (Blastp, KEGG) and gene names in a custom pathogenesis database (Table 5, FIGS. 6 & 7). The novel bacterial strain GW had genes in seven of the nine virulence-related gene clusters, however it had an incomplete type 1 secretion system (1 of 3 genes necessary for function) and no type III secretion system (0 of 10 genes necessary for function). These two secretion systems are important for the secretion of toxins and cell degrading enzymes into the host (type I), along with effectors (type III) (FIG. 6). The type III secretion system is complete in the pathogens X. translucens pv. translucens and X. translucnes pv. undulosa, whereas the type I secretion system is only in X. translucens pv. translucens (FIG. 7). The type III secretion system is known to be integral for virulence in X. translucens, as demonstrated in X. translucens pv. undulosa (Xtu4699) (Peng et al. 2016). The type III secretion system genes are either involved in the structure (Ysc/Hrc F, O, P, X, C, W, J, R, S, T, U, V, N, Q, L and HrpE) or the transport of effectors (Hrp B1, B2 and HpaT). These genes are normally localised in the genome of pathogenic Xanthomonas translucens strains (FIG. 8), but are completely absent in Xanthomonas sp. novel bacterial strain GW. There was also an absence of all conserved Type III effectors (XopAA, AD, AM, B, C2, F, G, K, N, O, V, X, Y, Z), variable Type III effectors (Xop, AF, AH, E1 L, P, AvrBs1, AvrBs2) and transcription activator-like effectors (TALEs 1-8) in Xanthomonas sp. novel bacterial strain GW. In pathogenic Xanthomonas strains these genes supress plant innate immunity and modulate plant cellular pathways to enhance bacterial infection (Buttner, 2016). Given that the Xanthomonas sp. novel bacterial strain GW did not have the type III secretion system and effectors it is thought that the strain is not a pathogen, but occupies an endophytic niche within perennial ryegrass. Furthermore, given that the Type III secretion system and effector genes are widely distributed across the whole chromosome of pahogenic Xanthomonas strains (FIG. 8), it is highly unlikely that Xanthomonas sp. novel bacterial strain GW would acquire all the genes necessary to become pathogenic through horizontal gene transfer.

TABLE 5
Virulence-related gene clusters identified in X. translucens pathovars

| | | Presence of genes |
|---|---|---|
| Virulence-related gene cluster | Reference | in strain GW |
| T1SS | Lee, S-W et al. (2006) | Incomplete |
| T2SS | Lee, H M et al. (2001) | Yes |
| T3SS | Wichmann et al. (2013) | No |
| T6SS | Boyer et al. (2009) | Yes |
| Type IV pilus | Dunger et al. (2016) | Yes |
| Flagellum | Darrasse et al. (2013) | Yes |
| Pathogenicity regulatory factors | Tang et al. (1991) | Yes |
| Xanthan biosynthesis | Katzen et al. (1996) | Yes |
| Lipopolysaccharide biosynthesis | Vorhölter, Niehaus | Yes |
| | and Pühler (2001) | |
| Conserved T3SS Effectors | Buttner (2016) | No |
| Variable T3SS Effectors | Buttner (2016) | No |
| Transcription activator-like | Cernadas et al., (2014); Hu et al., | No |
| Effectors | (2014) | |

### Example 6

In Planta Inoculations Supporting the Endophytic Niche of the Xanthomonas sp. Novel Bacterial Strains

To assess direct interactions between the Xanthomonas sp. novel bacterial strain GW and plants, an early seedling growth assay was established in barley. A total of 4 bacterial strains (GW—Xanthomonas sp.; Isolate 1, Isolate 2, Isolate 3—non Xanthomonads) were cultured in Lysogeny Broth (LB) overnight at 26° C. The following day seeds of barley (cultivar Hindmarsh) were surface-sterilised by soaking in 80% ethanol for 3 mins, then washing 5 times in sterile distilled water. The seeds were then soaked in the overnight cultures for 4 hours at 26° C. in a shaking incubator. For control seedlings, seeds were soaked in LB without bacteria for 4 hours at 26° C. in a shaking incubator. The seeds were planted into a pot trial, with three replicates (pots) per strain/control, with a randomised design. A total of 20 seeds were planted per pot, to a depth of 1 cm. The potting medium contained a mixture of 25% potting mix, 37.5% vermiculite and 37.5% perlite. The plants were grown for 5 days and then removed from the pots, washed, assessed for health (i.e. no disease symptoms) and photographed. The lengths of the longest root and the longest shoot were measured. Data was statistically analysed using a one-way ANOVA and Tukey test to detect the presence of any significant difference (p≤0.05) between treatments using OriginPro 2018 (Version b9.5.1.195).

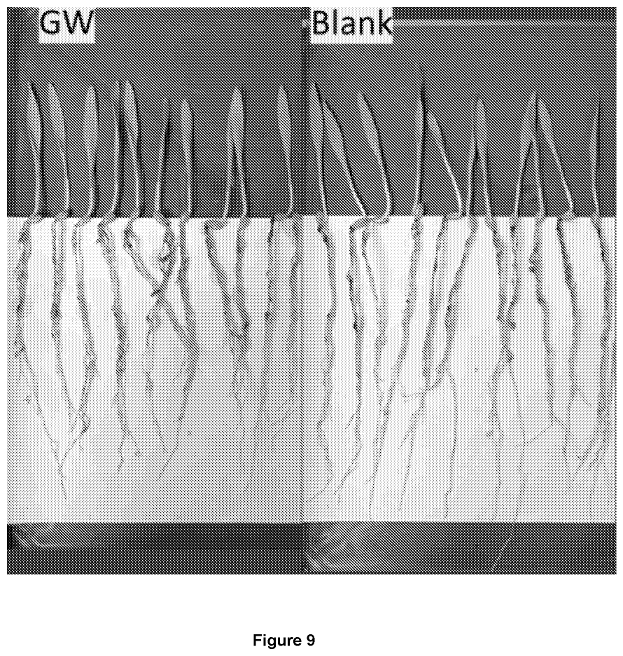

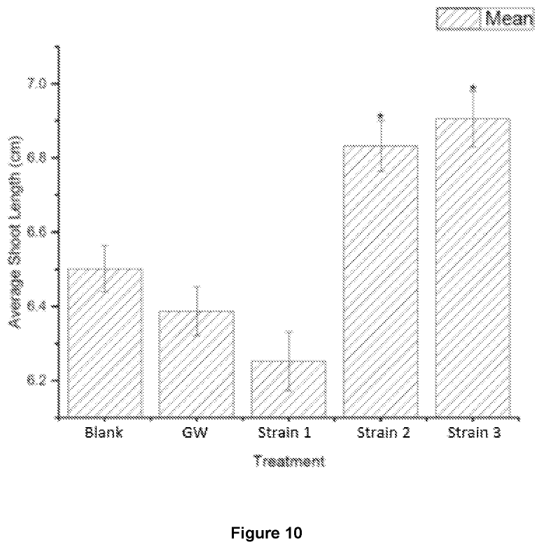

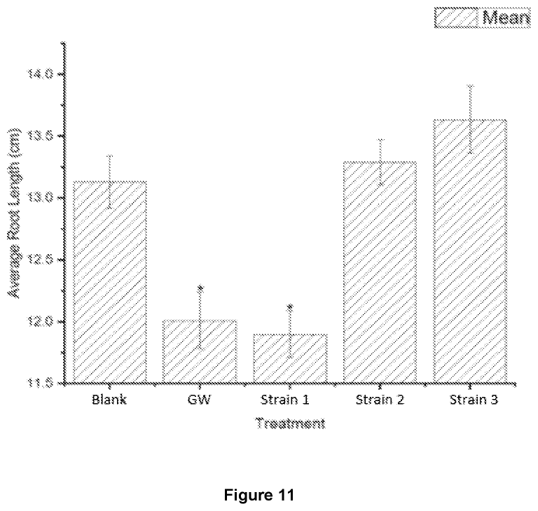

Seedlings inoculated with the Xanthomonas sp. novel bacterial strain GW were healthy with no disease symptoms recorded on leaves or roots (FIG. 9). The length of the shoots of seedlings inoculated with the Xanthomonas sp. novel bacterial strain GW were equivalent to the control (FIG. 10). The length of the roots of seedlings inoculated with the Xanthomonas sp. novel bacterial strain GW were significantly shorter than the control (FIG. 11).

### Example 7

In Planta Inoculations Supporting the Bioprotection Niche of the Xanthomonas sp. Novel Bacterial Strain GW

An in planta bioprotection assay was established in wheat to evaluate the activity of Xanthomonas sp. novel bacterial strain GW against the fungal phytopathogen Bipolaris sorokiniana (VPRI 42684). The bacterial strain was cultured in nutrient broth (BD Bioscience) for 6 hours. Wheat seeds were surface sterilised (3% NaOCl for 3 mins, 3× sterile dH2O wash), imbibed in bacterial culture for 18 hours, and then germinated in dark for 4 days for root and shoot development. Germinated seedlings were transferred in pots (4 seeds per pot, 4 pots per treatment) in a glasshouse for 39 days. A 7 cm segment of the lowest leaf that was green and fully extended from each plant was excised and placed on 0.5% water agar. A sterile sharp needle was used to create a wound at the centre of the leaf, to which 1 μL of B. sorokiniana spore suspension was added. Plates were then sealed and left at room temperature for 2 days. To assess the bioprotection activity, the size of lesion, chlorotic zones and fungal hyphal growth were recorded (measured in mm²). For the control, sterile Nutrient Broth was used. Statistical analysis (One-way ANOVA and Tukey Test) was conducted using OriginPro 2018 (Version b9.5.1.195) to detect the presence of any significant difference (P<0.05) between treatments.

Xanthomonas sp. novel bacterial strain GW significantly (P<0.05) reduced the average size of lesion and fungal hyphal growth compared to the control (Table 6). The lesion size was reduced by 96.7%, and the area of fungal hyphal growth was reduced by 94.7%.

TABLE 6
Bioprotection assay (in planta) results for Xanthomonas
sp. novel bacterial strain GW (average ± standard error)

| | | | Fungal hyphal |
|---|---|---|---|
| Isolate ID | Lesion/mm² | Chlorosis/mm² | growth/mm² |
| GW | 1.33 ± 0.25^{a} | 34.44 ± 10.72^{a} | 2.00 ± 1.37^{a} |
| Blank | 42.75 ± 10.26^{b} | 68.88 ± 22.50^{a} | 37.63 ± 20.45^{b} |

### Example 8

In Planta Inoculations Supporting Colonisation and Localisation of the Xanthomonas sp. Novel Bacterial Strain GW in Wheat and Perennial Ryegrass

Strain-specific primers were designed for Xanthomonas sp. novel bacterial strain GW targeting the 1997794 bp—2067075 bp region of the genome, which related to a section of the unique non-ribosomal peptide synthase of strain GW (GW-F CCACGCCGAATACAATGCAG; (SEQ ID NO 4) GW-R CATGGATGACTGGCACTGGT (SEQ ID NO 5); 5′→3′). An in silico analysis using Primer-BLAST and a sequence homology comparison to strain SS and SI indicated that the primers were strain-specific.

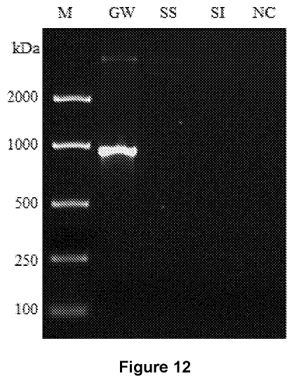

The strain-specific primer for GW was evaluated on cultures of strains Xanthomonas sp. novel bacterial strains GW, SS and SI. Initially, bacterial cultures were grown in nutrient broth (BD Bioscience) and grown overnight at 22° C. in the dark in a shaking incubator. The Promega Wizard® genomic DNA purification kit was used with the following modifications: initial centrifugation of 1 mL of overnight culture at 13,000-16,000×g for 2 mins was performed twice to pellet bacterial cells; incubations were conducted at −20° C. for 10 mins to enhance protein precipitation; DNA pellets were rehydrated in 50 mL rehydration solution at 65° C. for 10 mins followed by overnight incubation at 4° C. Final DNA concentration was measured using a Quantus™ Fluorometer and stored at 4° C. until further processing. The 25 μL reaction mixture contained: 12.5 μL of OneTaq™ Hot Start 2× master mix with standard buffer (New England BioLabs®), 2 μL of each primer (10 μM/μL), 8.5 μL of nuclease-free water and 2 μL of template DNA sample. The thermocycling conditions were: initial denaturation at 94° C. for 1 min, followed by 30 cycles of denaturation at 94° C. for 30 sec, annealing at 58° C. for 1 min, elongation at 72° C. for 2 min, and a final extension at 72° C. for 10 min. PCR products were separated at 120 V in a 2% (w/v) agarose gel containing 0.05 μL mL-1 SYBR safe stain in 1×TAE running buffer and visualized under UV light next to a 2 kb DNA ladder. The strain-specific primer generated an amplicon of the correct size (943 bp) for Xanthomonas sp. novel bacterial strain GW only (FIG. 12).

The strain-specific primer for GW was evaluated on wheat and perennial ryegrass plants inoculated with Xanthomonas sp. novel bacterial strain GW. Initially, perennial ryegrass and wheat seeds were sterilized in 70% ethanol for 3 minutes, followed by rinsing with sterilized distilled water (SDVV) for three times. The bacterial strain was cultured in nutrient broth (BD Bioscience) overnight, while seeds were imbibed in nutrient broth overnight in the dark. Seeds and the bacterial culture were combined for 4 hours in dark in a shaking incubator. For the controls, seeds were not inoculated with bacteria. A total of three seeds were sown per pot into potting mix and grown in a glasshouse. For perennial ryegrass, plants were harvested at three time points (12, 22 and 33 days after planting, DAP), while for wheat, plants were harvested at only one time point (7 DAP). For perennial ryegrass inoculated with GW, 20 replicates were maintained for each time point, while for wheat inoculated with GW 10 replicates were maintained. For the uninoculated control treatments (perennial ryegrass and wheat) 5 replicates were maintained for each time point. At harvest, plants were uprooted, washed thoroughly (roots only) and then sectioned into roots, pseudostem and leaves (ryegrass—12 & 22 DAP; wheat—7 DAP). However, for perennial ryegrass at 33 DAP, plants were sectioned into roots, pseudo-stem, lower leaves and upper leaves as plants were larger. Each section comprised three pieces (˜0.5 cm²) of plant tissue, which was placed into collection microtubes (2 mL) and stored at −80° C. The 22 and 33 DAP (perennial ryegrass) samples were freeze-dried for 48 hours, while the 7 (wheat) and 12 DAP (perennial ryegrass) samples were not freeze-dried. The Qiagen® MagAttract® 96 DNA plant core kit (Qiagen®, Hilden, Germany) was utilized to extract plant DNA using the Biomek® FXP lab automation workstation linked to Biomek software version v. 4.1 and Gen 5 (v. 2.08) software (Biotek Instruments, USA) with the following modifications to the manufacturer's instructions: to each well of the 96 well microplate, a 33 μL aliquot of RB buffer and 10 μL of resuspended MegAttract suspension G was added. A touch-down PCR (TD-PCR) was performed to enhance the sensitivity and specificity of primers in planta, compared to in vitro pure cultures. The PCR reaction mixture was prepared as per in vitro cultures. Touch-down PCR amplification was performed in two phases. In phase I, initial denaturation was carried out at 94° C. for 1 min, followed by 10 cycles of denaturation at 94° C. for 30 sec, annealing for at 65-55° C. (dropping 1C for each cycle) and 72° C. for 2 mins. In phase II, it was 20 cycles of denaturation at 94° C. for 30 sec, annealing at 58° C. for 1 min and extension at 72° C. for 2 min, with a final extension at 72° C. for 10 min. For perennial ryegrass, the presence of the Xanthomonas sp. novel bacterial strain GW was detected at 12, 22 and 33 DAP, with the highest rates of incidence recorded 22 DAP (20-85%) and the lowest at 7 DAP (0-1%) (Table 7). The most detections were recorded in consistently in roots (2-80%), followed by pseudostem (28-85%; 22 and 33 DAP only) and leaves (0-44%; 22 and 33 DAP only). There were no detections in the control. For wheat, the presence of the Xanthomonas sp. novel bacterial strain GW was detected at 7 DAP, with the highest rates of incidence recorded in roots (90%), followed by pseudostem (20%) and leaves (10%) (Table 8). Overall, Xanthomonas sp. novel bacterial strain GW appears to inoculate into both perennial ryegrass and wheat, where it colonises all tissues, but appears to preferentially colonise roots, and persists for at least 33 DAP.

TABLE 7
Incidence of GW in perennial ryegrass at three harvest time points.
The incidence is indicated as the number of plants showing the presence
of GW per total number of replicates inoculated or uninoculated (R—roots;
P—pseudostem; L—leaves; LL—lower leaves; UL—upper leaves).

| | 12 DAP | | | 22 DAP | | | 33 DAP | | | |
|---|---|---|---|---|---|---|---|---|---|---|
| | R | P | L | R | P | L | R | P | LL | UL |
| GW | 2/20 | 0/20 | 0/20 | 16/20 | 17/20 | 4/20 | 13/18 | 5/18 | 4/18 | 8/18 |
| Control | 0/5 | 0/5 | 0/5 | 0/5 | 0/5 | 0/5 | 0/5 | 0/5 | 0/5 | 0/5 |

TABLE 8
Incidence of GW in wheat at one harvest time point. The
incidence is indicated as the number of plants showing
the presence of GW per total number of replicates inoculated
or uninoculated (R—roots; P—pseudostem; L—leaves).

| | 7 DAP | | |
|---|---|---|---|
| | R | P | L |
| GW | 9/10 | 2/10 | 1/10 |
| Control | 0/5 | 0/5 | 0/5 |

### Example 9

In Planta Inoculations Supporting the Biofertilizer (Nitrogen) Niche of the Xanthomonas sp. Novel Bacterial Strain GW

An in planta biofertilizer assay was established in barley to evaluate the ability of Xanthomonas sp. novel bacterial strain GW to aid growth under nitrogen limiting conditions. Initially, bacterial strains (5, including GVV) were cultured in 20 mL nutrient broth (BD Bioscience) overnight at 26° C. whilst rotating at 200 RPM. The following day cultures were pelleted via centrifugation at 4000 RPM for 5 minutes, washed three times in 10 mL Phosphate Buffered Saline (PBS), resuspended in 20 mL PBS, quantified via spectrophotometry (OD600) and diluted (1:10). Barley seeds were sterilized in 70% ethanol for 5 minutes, followed by rinsing with sterilized distilled water (SDW) for five times. These sterile seeds were submerged in the dilution for 4 hours in a dark incubator at room temperature whilst rotating at 200 RPM. The seeds were subsequently transferred to moistened sterile filter paper and allowed to germinate for three days. The three-day-old seedlings were individually transferred to 60 mm plates with semi-solid Burks media (HiMedia) (5 g/L Agar). Seedlings were allowed to grow for a further 4 days, before the shoots and roots were measured for each seedling. There was a total of 6 treatments (5 bacterial strains including GW; 1 blank media control) containing 10 seedlings per treatment. Statistical analysis (One-way ANOVA and Tukey Test) was conducted using OriginPro 2018 (Version b9.5.1.195) to detect the presence of any significant difference (P<0.05) between treatments.

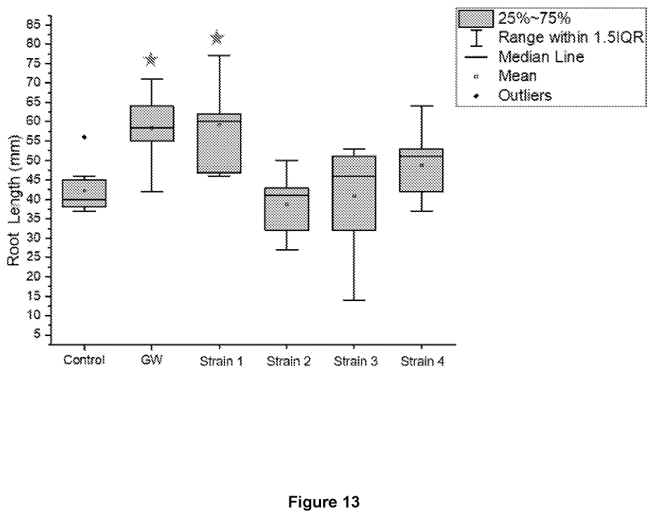

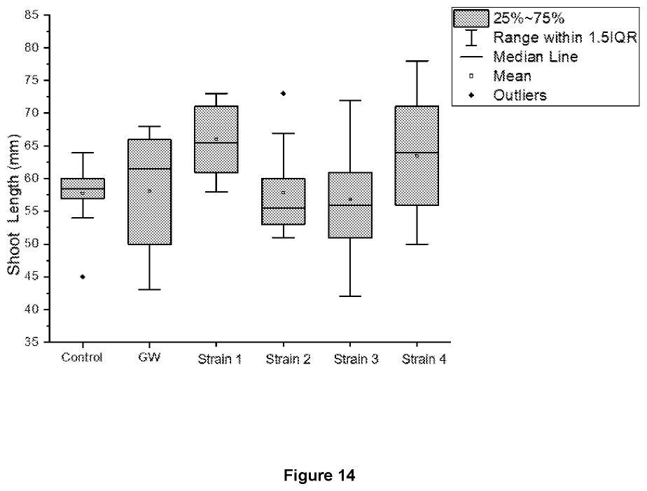

The root growth of seedlings inoculated with novel bacterial strain GW and grown under nitrogen limiting conditions was significantly greater than the control (P<0.05), with an average increase of 27.6% (FIG. 13). The shoot growth of seedlings inoculated with novel bacterial strain GW was not significantly greater than the control (P<0.05) (FIG. 14). Overall, results indicate that novel bacterial strain GW can aid in the growth of seedlings grown under nitrogen limiting conditions.

### Example 10

In Planta Inoculations Supporting the Biofertilizer (Phosphate Solubilisation) Niche of the Xanthomonas sp. Novel Bacterial strain GW

An in planta biofertilizer assay was established in barley to evaluate the ability of Xanthomonas sp. novel bacterial strain GW to aid growth under conditions with insoluble phosphate. Initially, bacterial strains (5, including GVV) were cultured in 30 mL R2B overnight at 26° C. whilst rotating at 200 RPM. The following day the barley seeds were sterilized in 70% ethanol for 5 minutes, followed by rinsing with SDW for five times. These sterile seeds were submerged in the overnight cultures for 4 hours in a dark incubator at room temperature whilst rotating at 200 RPM. The seeds were subsequently transferred to moistened sterile filter paper to be allowed to germinate for three days. These three-day-old seedlings were individually transferred to 60 mm plates with semi-solid Pikovskaya media which contains yeast extract (0.5 g/L), D-glucose (5.0 g/L), calcium phosphate (5.0 g/L), ammonium sulphate (0.5 g/L), potassium chloride (0.2 g/L), magnesium sulphate (0.1 g/L), manganese sulphate (0.1 mg/L), ferrous sulphate (0.1 mg/L) and agar (5.0 g/L). These seedlings were allowed to grow for another 4 days, before the shoots and roots were measured for each seedling. There was a total of 6 treatments (5 bacterial strains including GW; 1 blank media control) containing 10 seedlings per treatment. Statistical analysis (One-way ANOVA and Tukey Test) was conducted using OriginPro 2018 (Version b9.5.1.195) to detect the presence of any significant difference (P<0.05) between treatments.

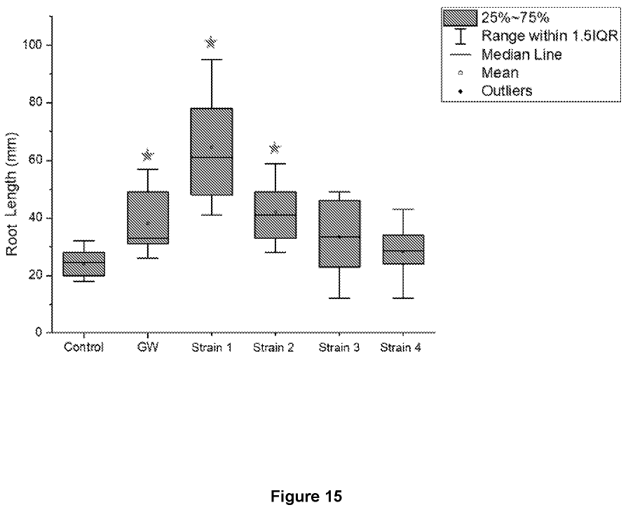

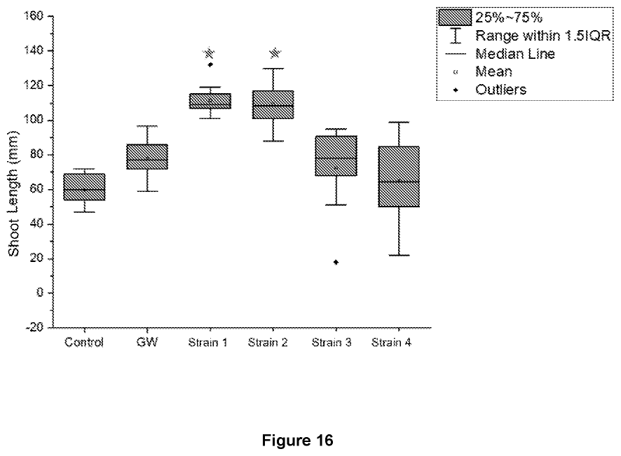

The root growth of seedlings inoculated with novel bacterial strain GW and grown under conditions with insoluble phosphate was significantly greater than the control (P<0.05), with an average increase of 36.5% (FIG. 15). The shoot growth of seedlings inoculated with novel bacterial strain GW was not significantly greater than the control (P<0.05) (FIG. 16). Overall, results indicate that novel bacterial strain GW can aid in the growth of seedlings grown under conditions with insoluble phosphate.

### Example 11

In Planta Inoculations Identifying Optimal Concentrations of Xanthomonas sp. Novel Bacterial Strain GW

An in planta biofertilizer assay was established in perennial ryegrass to evaluate the optimal concentration in which Xanthomonas sp. novel bacterial strain GW would support seedling growth. Initially, the bacterial strain was cultured overnight in 20 mL nutrient broth (BD Bioscience) at 26° C. whilst rotating at 200 RPM. The following day the culture was pelleted via centrifugation at 4000 RPM for 5 minutes, washed three times in 10 mL PBS, resuspended in 20 mL PBS, quantified via spectrophotometry (OD600). The culture was diluted (1:10) twice to create three concentrations (10⁰, 10⁻¹ and 10⁻²). The perennial ryegrass seeds were sterilized in 70% ethanol for 5 minutes, followed by rinsing five times with SDW. These sterile seeds were submerged in the dilutions for 4 hours in a dark incubator at room temperature whilst rotating at 200 RPM. After inoculation, 10 seeds were transferred to moistened sterile filter paper for germination from each dilution. After seven days, the roots and shoots were measured.

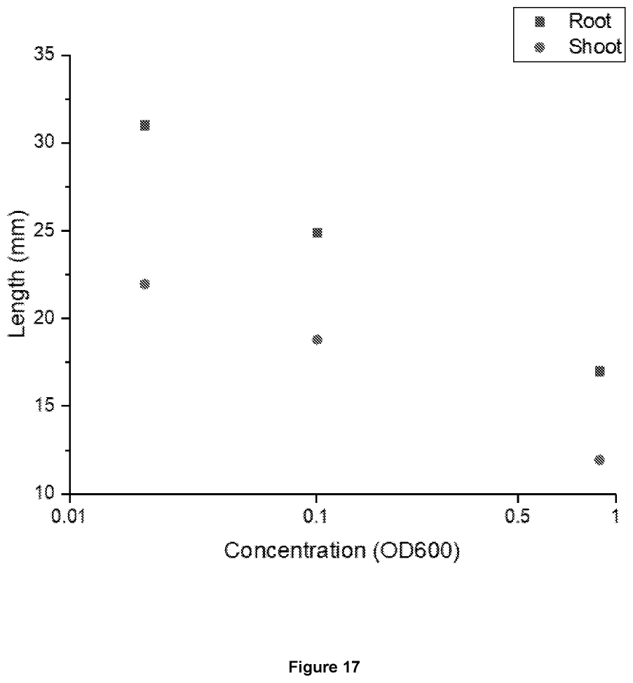

There was a trend observed whereby root and shoot growth increased as the concentration of novel bacteria GW decreased (FIG. 17). The greatest root growth was observed at the 10⁻² dilution, which was 19.7% greater than 10⁻¹ dilution and 45.2% greater than the 10⁻⁰ dilution. The greatest shoot growth was observed at the 10⁻² dilution, which was 14.5% greater than 10⁻¹ dilution and 45.9% greater than the 10⁻⁰ dilution.

It is to be understood that various alterations, modifications and/or additions may be made without departing from the spirit of the present invention as outlined herein.

As used herein, except where the context requires otherwise, the term “comprise” and variations of the term, such as “comprising”, “comprises” and “comprised”, are not intended to be in any way limiting or to exclude further additives, components, integers or steps.

Reference to any prior art in the specification is not, and should not be taken as, an acknowledgment or any form of suggestion that this prior art forms part of the common general knowledge in Australia or any other jurisdiction or that this prior art could reasonably be expected to be combined by a person skilled in the art.

### REFERENCES

1. Ankenbrand, M J, Hohlfeld, S, Hackl, T & Förster, F 2017, ‘AliTV—interactive visualization of whole genome comparisons’, PeerJ Computer Science, vol. 3.

2. Bolger, A M, Lohse, M & Usadel, B 2014, ‘Trimmomatic: a flexible trimmer for Illumina sequence data’, Bioinformatics, vol. 30, no. 15, pp. 2114-20.

3. Boyer, F, Fichant, G, Berthod, J, Vandenbrouck, Y & Attree, I 2009, ‘Dissecting the bacterial type VI secretion system by a genome wide in silico analysis: what can be learned from available microbial genomic resources?’, BMC genomics, vol. 10, p. 104.

4. Buttner, D. 2016. Behind the lines-actions of bacterial type III effector proteins in plant cells. FEMS Microbiol Rev 40, 894-937.

5. Cernadas, R. A., Doyle, E. L., Nino-Liu, D. O., Wilkins, K. E., Bancroft, T., Wang, L., Schmidt, C. L., Caldo, R., Yang, B., White, F. F., Nettleton, D., Wise, R. P., and Bogdanove, A. J. 2014. Code-assisted discovery of TAL effector targets in bacterial leaf streak of rice reveals contrast with bacterial blight and a novel susceptibility gene. PLoS Pathog 10, e1003972.

6. Darrasse, A, Carrère, S, Barbe, V, Boureau, T, Arrieta-Ortiz, M L, Bonneau, S, Briand, M, Brin, C, Cociancich, S, Durand, K, Fouteau, S, Gagnevin, L, Guerin, F, Guy, E, Indiana, A, Koebnik, R, Lauber, E, Munoz, A, Noel, L D, Pieretti, I, Poussier, S, Pruvost, O, Robène-Soustrade, I, Rott, P, Royer, M, Serres-Giardi, L, Szurek, B, van Sluys, M-A, Verdier, V, Vernière, C, Arlat, M, Manceau, C & Jacques, M-AJBG 2013, ‘Genome sequence of Xanthomonas fuscans subsp. fuscansstrain 4834-R reveals that flagellar motility is not a general feature of xanthomonads’, vol. 14, no. 1, p. 761.

7. De Coster, W, D'Hert, S, Schultz, D T, Cruts, M & Van Broeckhoven, C 2018, ‘NanoPack: visualizing and processing long read sequencing data’, Bioinformatics.

8. Dunger, G, Llontop, E, Guzzo, C R & Farah, C S 2016, ‘The Xanthomonas type IV pilus’, Curr Opin Microbiol, vol. 30, pp. 88-97.

9. Katzen, F, Becker, A, Zorreguieta, A, Pühler, A & lelpi, L J Job 1996, ‘Promoter analysis of the Xanthomonas campestris pv. campestris gum operon directing biosynthesis of the xanthan polysaccharide’, vol. 178, no. 14, pp. 4313-8.

10. Hu, Y., Zhang, J., Jia, H., Sosso, D., Li, T., Frommer, W. B., Yang, B., White, F. F., Wang, N., and Jones, J. B. 2014. Lateral organ boundaries 1 is a disease susceptibility gene for citrus bacterial canker disease. Proc Natl Acad Sci U S A 111, E521-529.

11. Lee, H M, Tyan, S W, Leu, W M, Chen, L Y, Chen, D C & Hu, N T 2001, ‘Involvement of the XpsN protein in formation of the XpsL-xpsM complex in Xanthomonas campestris pv. campestris type II secretion apparatus’, J Bacteriol, vol. 183, no. 2, pp. 528-35.

12. Lee, S-W, Han, S-W, Bartley, L E & Ronald, PCJPotNAoS 2006, ‘Unique characteristics of Xanthomonas oryzae pv. oryzae AvrXa21 and implications for plant innate immunity’, vol. 103, no. 49, pp. 18395-400.

13. Löytynoja, A 2014, ‘Phylogeny-aware alignment with PRANK’, in D J Russell (ed.), Multiple Sequence Alignment Methods, Humana Press, Totowa, N.J., pp. 155-70, DOI 10.1007/978-1-62703-646-7_10, <https://doi.orq/10.1007/978-1-62703-646-7 10>.

14. Page, A J, Cummins, C A, Hunt, M, Wong, V K, Reuter, S, Holden, M T, Fookes, M, Falush, D, Keane, J A & Parkhill, J 2015, ‘Roary: rapid large-scale prokaryote pan genome analysis’, Bioinformatics, vol. 31, no. 22, pp. 3691-3.

15. Peng, Z, Hu, Y, Xie, J, Potnis, N, Akhunova, A, Jones, J, Liu, Z, White, F F & Liu, S 2016, ‘Long read and single molecule DNA sequencing simplifies genome assembly and TAL effector gene analysis of Xanthomonas translucens’, BMC genomics, vol. 17, p. 21.

16. Price, M N, Dehal, P S & Arkin, A P 2010, ‘FastTree 2-approximately maximum-likelihood trees for large alignments’, PLoS One, vol. 5, no. 3, p. e9490.

17. Seemann, T 2014, ‘Prokka: rapid prokaryotic genome annotation’, Bioinformatics, vol. 30, no. 14, pp. 2068-9.

18. Tang, J-L, Liu, Y-N, Barber, C E, Dow, J M, Wootton, J C, Daniels, MJJM & MGG, GG 1991, ‘Genetic and molecular analysis of a cluster of rpf genes involved in positive regulation of synthesis of extracellular enzymes and polysaccharide in Xanthomonas campestris pathovar campestris’, vol. 226, no. 3, pp. 409-17.

19. Vaser, R, Sovic, I, Nagarajan, N & Sikic, M 2017, ‘Fast and accurate de novo genome assembly from long uncorrected reads’, Genome Res, vol. 27, no. 5, pp. 737-46.

20. Vorhölter, F-J, Niehaus, K & Pühler, A 2001, ‘Lipopolysaccharide biosynthesis in Xanthomonas campestris pv. campestris: a cluster of 15 genes is involved in the biosynthesis of the LPS O-antigen and the LPS core’, Molecular Genetics and Genomics, vol. 266, no. 1, pp. 79-95.

21. Walker, B J, Abeel, T, Shea, T, Priest, M, Abouelliel, A, Sakthikumar, S, Cuomo, C A, Zeng, Q, Wortman, J, Young, S K & Earl, A M 2014, ‘Pilon: an integrated tool for comprehensive microbial variant detection and genome assembly improvement’, PLoS One, vol. 9, no. 11, p. e112963.

22. Weber, T, Blin, K, Duddela, S, Krug, D, Kim, H U, Bruccoleri, R, Lee, S Y, Fischbach, M A, Muller, R, Wohlleben, W, Breitling, R, Takano, E & Medema, M H 2015, ‘antiSMASH 3.0-a comprehensive resource for the genome mining of biosynthetic gene clusters’, Nucleic Acids Res, vol. 43, no. W1, pp. W237-43.

23. Wichmann, F, Vorholter, F J, Hersemann, L, Widmer, F, Blom, J, Niehaus, K, Reinhard, S, Conradin, C & Kolliker, R 2013, ‘The noncanonical type III secretion system of Xanthomonas translucens pv. graminis is essential for forage grass infection’, Mol Plant Pathol, vol. 14, no. 6, pp. 576-88.

24. Wick, R R, Judd, L M, Gorrie, C L & Holt, K E 2017, ‘Unicycler: Resolving bacterial genome assemblies from short and long sequencing reads’, PLOS Computational Biology, vol. 13, no. 6, p. e1005595.

25. Wick, R R, Schultz, M B, Zobel, J & Holt, K E 2015, ‘Bandage: interactive visualization of de novo genome assemblies’, Bioinformatics, vol. 31, no. 20, pp. 3350-2.

1-23. (canceled)

24. A substantially purified or isolated endophyte strain isolated from a plant of the Poaceae family, wherein said endophyte is a strain of Xanthomonas sp. which provides bioprotection and/or biofertilizer phenotypes to plants into which it is inoculated.

25. The endophyte according to claim 24, wherein the bioprotection and/or biofertilizer phenotype includes production of the bioprotectant compound in the plant into which the endophyte is inoculated.

26. The endophyte according to claim 25, wherein the bioprotectant compound is selected from the group consisting of siderophore xanthoferrin, and/or xanthomonadin, or a derivative, isomer and/or salt thereof.

27. The endophyte according to claim 24, wherein the bioprotection and/or biofertilizer phenotype is selected from the group consisting of production of organic acids, solubilisation of phosphate and nitrogen fixation in the plant into which the endophyte is inoculated.

28. The endophyte according to claim 24, wherein the endophyte is strain is selected from the group consisting of Xanthomonas sp. strains GW, SS and SI as deposited with The National Measurement Institute on 17 May 2019 with accession numbers V19/009902, V19/009905 and V19/009909, respectively.

29. The endophyte according to claim 24, wherein the plant from which the endophyte is isolated is a pasture grass.

30. The endophyte according to claim 29, wherein the pasture grass is from the genus Lolium or Festuca.

31. The endophyte according to claim 30, wherein the pasture grass is from the species Lolium perenne or Festuca arundinaceum.

32. The endophyte according to claim 24, wherein the plant into which the endophyte is inoculated includes an endophyte-free host plant or part thereof stably infected with said endophyte.

33. The endophyte according to claim 24, wherein the plant into which the endophyte is inoculated is an agricultural plant species selected from one or more of forage grass, turf grass, bioenergy grass, grain crop and industrial crop.

34. The endophyte according claim 33, wherein the plant into which the endophyte is inoculated is a forage, turf or bioenergy grass selected from the group consisting of those belonging to the genera Lolium and Festuca, including L. perenne (perennial ryegrass), L. arundinaceum (tall fescue) and L. multiflorum (Italian ryegrass), and those belonging to the Brachiaria-Urochloa species complex (panic grasses), including Brachiaria brizantha, Brachiaria decumbens, Brachiaria humidicola, Brachiaria stolonifera, Brachiaria ruziziensis, B. dictyoneura, Urochloa brizantha, Urochloa decumbens, Urochloa humidicola, Urochloa mosambicensis as well as interspecific and intraspecific hybrids of Brachiaria-Urochloa species complex such as interspecific hybrids between Brachiaria ruziziensis x Brachiaria brizantha, Brachiaria ruziziensis x Brachiaria decumbens, [Brachiaria ruziziensis x Brachiaria decumbens]x Brachiaria brizantha, [Brachiaria ruziziensis x Brachiaria brizantha] x Brachiaria decumbens.

35. The endophyte according claim 33, wherein the plant into which the endophyte is inoculated is a grain crop or industrial crop grass selected from the group consisting of those belonging to the genus Triticum, including T. aestivum (wheat), those belonging to the genus Hordeum, including H. vulgare (barley), those belonging to the genus Avena, including A. sativa (oats), those belonging to the genus Zea, including Z. mays (maize or corn), those belonging to the genus Oryza, including O. sativa (rice), those belonging to the genus Saccharum including S. officinarum (sugarcane), those belonging to the genus Sorghum including S. bicolor (sorghum), those belonging to the genus Panicum, including P. virgatum (switchgrass), those belonging to the genera Miscanthus, Paspalum, Pennisetum, Poa, Eragrostis and Agrostis.

36. The endophyte according to claim 33, wherein the plant into which the endophyte is inoculated is a grain crop or industrial crop selected from the group consisting of wheat, barley, oats, chickpeas, triticale, fava beans, lupins, field peas, canola, cereal rye, vetch, lentils, millet/panicum, safflower, linseed, sorghum, sunflower, maize, canola, mungbeans, soybeans, and cotton.

37. A plant or part thereof infected with one or more endophytes according to claim 24.

38. A bioprotectant compound produced by an endophyte according to claim 24, or a derivative, isomer and/or a salt thereof.

39. A bioprotectant compound according to claim 38, wherein the compound is selected from siderophore xanthoferrin and xanthomonadin, or a derivative, isomer and/or salt thereof.

40. A method for producing a bioprotectant compound, said method including infecting a plant with the endophyte according to claim 24 and cultivating the plant under conditions suitable to produce the bioprotectant compound.

41. The method according to claim 40, wherein the conditions include a culture medium including a source of carbohydrates, preferably wherein the source of carbohydrates is selected from one or more of the group consisting of a starch/sugar-based agar or broth, a cereal-based agar or broth, endophyte agar, Murashige and Skoog with 20% sucrose, half V8 juice/half PDA, water agar and yeast malt extract agar.

42. The method according to claim 40, wherein the method further includes isolating the bioprotectant compound from the plant or culture medium.

43. The method according to claim 42, wherein the bioprotectant compound is selected from siderophore xanthoferrin and xanthomonadin, or a derivative, isomer and/or salt thereof.


SEQUENCE LISTING (5 sequences)

SEQ ID NO: 1 - DNA, 1269 bp, Xanthomonas sp.
tctaccttttcgtgggggataacgtagggaaacttacgctaataccgcatacgaccttagggtgaaagcggaggaccttcgggcttcgcgcggatagatgagccgatgtcggattagctagttggcggggtaaaggcccaccaaggcgacgatccgtagctggtctgagaggatgatcagccacactggaactgagacacggtccagactcctacgggaggcagcagtggggaatattggacaatgggcgcaagcctgatccagccatgccgcgtgggtgaagaaggccttcgggttgtaaagcccttttgttgggaaagaaaagcagtcggttaatacccgattgttctgacggtacccaaagaataagcaccggctaacttcgtgccagcagccgcggtaatacgaagggtgcaagcgttactcggaattactgggcgtaaagcgtgcgtaggtggttgtttaagtccgttgtgaaagccctgggctcaacctgggaattgcagtggatactgggcaactagagtgtggtagaggatggcggaattcccggtgtagcagtgaaatgcgtagagatcgggaggaacatctgtggcgaaggcggccatctggaccaacactgacactgaggcacgaaagcgtggggagcaaacaggattagataccctggtagtccacgccctaaacgatgcgaactggatgttgggtgcaacttggcacgcagtatcgaagctaacgcgttaagttcgccgcctggggagtacggtcgcaagactgaaactcaaaggaattgacgggggcccgcacaagcggtggagtatgtggtttaattcgatgcaacgcgaagaaccttacctggtcttgacatccacggaactttccagagatggattggtgccttcgggaaccgtgagacaggtgctgcatggctgtcgtcagctcgtgtcgtgagatgttgggttaagtcccgcaacgagcgcaacccttgtccttagttgccagcacgtaatggtgggaactctaaggagaccgccggtgacaaaccggaggaaggtggggatgacgtcaagtcatcatggcccttacgaccagggctacacacgtactacaatggttaggacagagggctgcaaactcgcgagagtaagccaatcccagaaacctaatctcagtccggattggagtctgcaactcgactccatgaagtcggaatcgctagtaatcgcagatcagcattgctgcggtgaatacgttcccgggccttgtaca

SEQ ID NO: 2 - DNA, 20 bp, Artificial Sequence
  Primer
agagtttgatcmtggctcag

SEQ ID NO: 3 - DNA, 19 bp, Artificial Sequence
  Primer
ggttaccttgttacgactt

SEQ ID NO: 4 - DNA, 20 bp, Artificial Sequence
  Primer
ccacgccgaatacaatgcag

SEQ ID NO: 5 - DNA, 20 bp, Artificial Sequence
  Primer
catggatgactggcactggt